(19) United States
(12) Patent Application Publication
(10) Pub. No.: US 2018/0299468 A1
(43) Pub. Date: Oct. 18, 2018

(54) Method for Diagnosing Lactation Sufficiency

(71) Applicant: The Penn State Research Foundation, University Park, PA (US)

(72) Inventor: Shannon L. Kelleher, Harrisburg, PA (US)

(21) Appl. No.: 15/766,889

(22) PCT Filed: Oct. 10, 2016
(86) PCT No.: PCT/US2016/056243

§ 371 Date: Apr. 9, 2018

Related U.S. Application Data
(60) Provisional application No. 62/238,914, filed on Oct. 8, 2015.

(51) Int. Cl. G01N 33/68; C12Q 1/6883

(52) U.S. Cl. CPC G01N 33/6893; G01N 2800/365; C12Q 2600/158; C12Q 1/6883

(57) ABSTRACT

The present invention provides for the first time the identification of breastmilk exosomal biomarkers useful in diagnosing lactation insufficiency. The present invention therefore provides methods, kits and systems for diagnosing lactation insufficiency, by examining relevant proteins and RNA in exosomes isolated from a patient's breastmilk.

### CROSS-REFERENCE TO RELATED APPLICATIONS

This application claims priority to U.S. Provisional Application Ser. No. 62/238,914, filed Oct. 8, 2015, the contents of which are incorporated by reference herein in its entirety.

### BACKGROUND OF THE INVENTION

Breast milk is best, but only if a woman is producing enough, high-quality milk. It has been estimated that ˜1-5% of women suffer from lactation failure, but experts believe this number to be much higher and may be closer to 15%. Additionally, the number of women producing sub-optimal breast milk (herein referred to as “lactation insufficiency”) as a result of genetics, maternal health, diet, environmental exposures or other unknown factors is not known, although countless women are concerned that they are not making enough milk to meet their infant's needs, or that their infant is “allergic” to something in their milk. In fact, 33% of women wean their children prior to 6 months of age (the current recommendation of the American Academy of Pediatrics) due to concerns regarding lactation insufficiency (e.g., low volume or sub-optimal composition). There is currently no way to diagnose lactation insufficiency, other than noting the decline in a breastfed infant's health.

Exosomes and other microvesicles are found in biological fluids including plasma, milk, urine and saliva and are believed to reflect the function of the cell of origin. Exosomes are microvesicles that contain cytoplasmic molecules (proteins, microRNAs, organic molecules) surrounded by membrane and proteins derived from intracellular compartments. Globules (specialized microvesicles found in milk) contain cytoplasmic molecules and lipid surrounded by membrane and proteins derived from secretory vesicles and apical membrane of the secreting mammary epithelial cell. Molecular profiles of exosomes isolated from blood plasma are currently being exploited in the development of novel cancer diagnostics.

Molecules within microvesicles such as exosomes and globules reflect the function of the cell of origin and can be used as bioreporters to report lactation insufficiency. Many studies show that profound physiological and metabolic changes occur in the liver, muscle and mammary gland during lactation in order to produce milk. In addition, a recent report suggests that physiological changes that occur during exercise lead to changes in secretion from the salivary glands. Exosomes in plasma and urine may report these metabolic shifts in liver and muscle, exosomes and globules in milk may report the function of the mammary gland, and exosomes in saliva may report changes in the function of the salivary gland. The goal is to use the molecular profile of exosome and globule proteins to develop novel diagnostics tools to identify women to suffer from lactation insufficiency so that healthcare providers can intervene prior to the onset of illness in their infant.

Thus, there is an urgent need in the art for compositions and methods for diagnosing lactation (in)sufficiency prior to the point in which infant health is compromised or they are prematurely weaned. The present invention addresses these needs.

### SUMMARY OF THE INVENTION

In one aspect, the present invention provides a method of diagnosing a lactation performance of a subject. In one embodiment the method comprises a) isolating microvesicles in a biological sample of the subject; b) determining the level of a biomarker in the microvesicles; c) comparing the level of the biomarker in the biological sample of the subject with a comparator control; and d) diagnosing the lactation performance of the subject to be lactation insufficient if the level of the biomarker in the biological sample of the subject is altered at a statistically significant amount when compared with the level of the biomarker of the comparator control.

In one embodiment, the microvesicles are selected from the group consisting of exosomes and globules. In one embodiment, biological sample is selected from the group consisting of breastmilk, urine, saliva and plasma.

In one embodiment, the biomarker is selected from the group consisting of vitronectin, folate receptor alpha, peroxiredoxin-6, mucin-4, elongation factor 2, cytosolic 10-F-D, endoplasmin, and any combination thereof.

In one embodiment, the lactation insufficiency is selected from the group consisting of low volume, sub-optimal composition, and a combination thereof.

In one embodiment, the comparator control is the level of the biomarker in the biological sample of a lactation sufficient subject. In one embodiment, the level of the biomarker in the biological sample of subject is increased at a statistically significant amount when compared with the level of the biomarker of the comparator control. In another embodiment, the level of the biomarker in the biological sample of subject is decreased at a statistically significant amount when compared with the level of the biomarker of the comparator control. In one embodiment, the comparator control is at least one selected from the group consisting of: a positive control, a negative control, a historical control, a historical norm, or the level of a reference molecule in the biological sample.

In one embodiment, the level of the biomarker in the biological sample is determined by measuring the level of polypeptide of the biomarker in the biological sample. In one embodiment, the level of the biomarker in the biological sample is determined by measuring the level of mRNA of the biomarker in the biological sample.

The present invention also provides a method of diagnosing lactation insufficiency in a subject. In one embodiment the method comprises a) isolating microvesicles in a biological sample of the subject; b) determining the level of a biomarker in the microvesicles; c) comparing the level of the biomarker in the biological sample of the subject with a comparator control; and d) diagnosing the lactation performance of the subject to be lactation insufficient if the level of the biomarker in the biological sample of the subject is altered at a statistically significant amount when compared with the level of the biomarker of the comparator control.

In another aspect, the present invention provides a system for diagnosing lactation performance. In one embodiment, the system detects the level of a biomarker in microvesicles isolated from a biological sample obtained from a subject, wherein the of the biomarker diagnoses lactation insufficiency in the subject.

In one embodiment, the microvesicles are selected from the group consisting of exosomes and globules. In one embodiment, biological sample is selected from the group consisting of breastmilk, urine, saliva and plasma.

In one embodiment, the biomarker is selected from the group consisting of vitronectin, folate receptor alpha, peroxiredoxin-6, mucin-4, elongation factor 2, cytosolic 10-F-D, endoplasmin, and any combination thereof.

In one embodiment, the subject is human.

In one embodiment, the reagent is selected from the group consisting of a microfluidics chip and a lateral flow test.

The present invention also provides a system for diagnosing lactation insufficiency. In one embodiment, the system detects the level of a biomarker in microvesicles isolated from a biological sample obtained from a subject, wherein the of the biomarker diagnoses lactation insufficiency in the subject.

In yet another aspect, the present invention provides a kit for diagnosing lactation performance. In one embodiment the kit comprises a reagent for measuring the level of a biomarker in microvesicles isolated from a biological sample of a subject. In one embodiment, the biomarker is selected from the group consisting of vitronectin, folate receptor alpha, peroxiredoxin-6, mucin-4, elongation factor 2, cytosolic 10-F-D, endoplasmin, and any combination thereof. In one embodiment, the microvesicles are selected from the group consisting of exosomes and globules.

In one embodiment, biological sample is selected from the group consisting of breastmilk, urine, saliva and plasma. In one embodiment, the subject is human.

The invention also provides a kit for diagnosing lactation insufficiency. In one embodiment, the kit comprises a reagent for measuring the level of a biomarker in microvesicles isolated from a biological sample of a subject.

### DETAILED DESCRIPTION

The present invention provides biomarkers in easily accessible fluids including plasma, urine, saliva and breast milk that report lactation insufficiency. The markers of the invention can be used to screen, diagnose and monitor lactation insufficiency. The markers of the invention can be used to establish and evaluate treatment plans to monitor lactation insufficiency.

The present invention therefore provides compositions and methods of diagnosing lactation insufficiency, by examining relevant biomarkers and their expression. In one embodiment, the biomarker is found in microvesicles in a biological sample of the subject. In one embodiment, the biomarker is found in microvesicles isolated from a biological sample of the subject. In another embodiment, the biological sample is breastmilk, urine, saliva or plasma. In one embodiment the microvesicle is an exosome or a globule. In one embodiment, biomarker expression includes transcription into messenger RNA (mRNA) and translation into protein, as well as transcription into types of RNA such as transfer RNA (tRNA), ribosomal RNA (rRNA) and microRNA that are not translated into protein.

In one embodiment, the invention provides biomarkers for diagnosing the lactation performance of a subject. In one embodiment, the invention provides a biomarker for the detection of lactation insufficiency. In one embodiment, the biomarker for the detection of lactation insufficiency includes but is not limited to vitronectin, folate receptor alpha, peroxiredoxin-6, mucin-4, elongation factor 2, cytosolic 10-F-D, and endoplasmin.

In some embodiments, the biomarker is selected from 14-3-3 protein, 40S ribosomal protein, 60S ribosomal protein, 60S ribosomal protein L3, 60S ribosomal protein L5, 60S ribosomal protein L7, Annexin A2, Apolipoprotein E, Carbonic Anhydrase, CD14, Cell division control protein 42, Chordin-like protein 2, Complement C3, Cytosolic 10-F-D, Elongation Factor 1, Elongation Factor 2, Endoplasmin, ER ATPase, Ezrin, Fibronectin, Folate receptor alpha, Gelsolin, Hyaluronan and proteoglycan link protein, Immunoglobulin J chain, Keratin 2, Keratin 5, Keratin 6A, Keratin 14, Keratin 16, Keratin 17, Lactoperoxidase, Lanosterol Synthase, Lysozyme C, Mucin-1, Mucin-4, Neutral alpha-glucosidase, Peptidyl-prolyl cis-trans isomerase A, Peroxiredoxin-6, Protein disulfide isomerase, Serum Albumin, Sulfhydryl oxidase 1, Syntaxin binding protein 2, Tenascin, Toll-like receptor 2, and Vitronectin.

Accordingly, in some embodiments of the invention, a method for diagnosing the lactation performance of a subject is provided. In certain non-limiting embodiments, a method for diagnosing lactation insufficiency in a subject is provided. The method comprises a) providing a biological sample from the subject; b) isolating microvesicles from the biological sample; c) analyzing the microvesicles with an assay that specifically detects at least one biomarker of the invention in the isolated microvesicles; d) comparing the subject biomarker profile with a control biomarker profile wherein a statistically significant difference between the subject biomarker profile and the control biomarker profile is indicative of lactation insufficiency; and e) effectuating a treatment regimen based thereon.

In some embodiments, the method comprises a) providing a biological sample from the subject b) analyzing microvesicles in the biological sample with an assay that specifically detects at least one biomarker of the invention in the microvesicles; d) comparing the subject biomarker profile with a control biomarker profile wherein a statistically significant difference between the subject biomarker profile and the control biomarker profile is indicative of lactation insufficiency; and e) effectuating a treatment regimen based thereon.

In one embodiment, the biomarker types comprise mRNA or microRNA biomarkers. In one embodiment, the mRNA or microRNA is detected by mass spectroscopy, PCR, microarray, thermal sequencing, capillary array sequencing, solid phase sequencing, and the like.

In another embodiment, the biomarker types comprise polypeptide biomarkers. In one embodiment, the polypeptide is detected by ELISA, Western blot, flow cytometry, immunofluorescence, immunohistochemistry, mass spectroscopy, and the like.

In some embodiments of the invention, a system for diagnosing lactation performance is provided. In certain non-limiting embodiments, a system for diagnosing lactation insufficiency is provided. The system detects the level of a biomarker in microvesicles in a biological sample of a subject, wherein the of the biomarker diagnoses lactation insufficiency in the subject. In one embodiment, the microvesicles are exosomes or globules. In another embodiment, the biological sample is breastmilk, urine, saliva or plasma. In one embodiment, the biomarker includes but is not limited to vitronectin, folate receptor alpha, peroxiredoxin-6, mucin-4, elongation factor 2, cytosolic 10-F-D, and endoplasmin.

In various embodiments, the biomarker is selected from 14-3-3 protein, 40S ribosomal protein, 60S ribosomal protein, 60S ribosomal protein L3, 60S ribosomal protein L5, 60S ribosomal protein L7, Annexin A2, Apolipoprotein E, Carbonic Anhydrase, CD14, Cell division control protein 42, Chordin-like protein 2, Complement C3, Cytosolic 10-F-D, Elongation Factor 1, Elongation Factor 2, Endoplasmin, ER ATPase, Ezrin, Fibronectin, Folate receptor alpha, Gelsolin, Hyaluronan and proteoglycan link protein, Immunoglobulin J chain, Keratin 2, Keratin 5, Keratin 6A, Keratin 14, Keratin 16, Keratin 17, Lactoperoxidase, Lanosterol Synthase, Lysozyme C, Mucin-1, Mucin-4, Neutral alpha-glucosidase, Peptidyl-prolyl cis-trans isomerase A, Peroxiredoxin-6, Protein disulfide isomerase, Serum Albumin, Sulfhydryl oxidase 1, Syntaxin binding protein 2, Tenascin, Toll-like receptor 2, and Vitronectin.

In some embodiments, the subject is a mammal. In certain non-limiting embodiments, the subject is a human.

In some instances, the system of the invention may take the form of a user-friendly point-of-use platform, for example a lateral flow device, having a sample application region and a readable detection region to indicate the presence or absence of the biomarker or variable levels of the biomarker.

In one embodiment, the system of the invention detects a biomarker by way of a lateral flow immunoassay that utilizes strips of cellulose membrane onto which antibodies and other reagents are applied. For example, the test sample moves along the strip due to capillary action and reacts with the reagents at different points along the strip. The end result is the appearance or absence of a detectable line or spot.

In one embodiment, the lateral flow device can be in the form of a cartridge that can be read by a machine. Preferably, the machine is automated.

In one embodiment, the system of the invention detects a biomarker by way of a point-of-care platform, for example a microfluidics-based chip.

In yet another embodiment, the reagent is a microfluidics chip or a lateral flow test.

In some aspects, the invention provides a kit for diagnosing lactation performance. In certain non-limiting embodiments, the kit diagnoses lactation insufficiency. In one embodiment, the kit comprises a reagent for measuring the level of a biomarker in microvesicles isolated from a biological sample of a subject. In one embodiment, the kit comprises a reagent for measuring the level of a biomarker in microvesicles in a biological sample of a subject.

In one embodiment, the biomarker is vitronectin, folate receptor alpha, peroxiredoxin-6, mucin-4, elongation factor 2, cytosolic 10-F-D, endoplasmin, or any combination thereof.

In various embodiments, the biomarker is selected from 14-3-3 protein, 40S ribosomal protein, 60S ribosomal protein, 60S ribosomal protein L3, 60S ribosomal protein L5, 60S ribosomal protein L7, Annexin A2, Apolipoprotein E, Carbonic Anhydrase, CD14, Cell division control protein 42, Chordin-like protein 2, Complement C3, Cytosolic 10-F-D, Elongation Factor 1, Elongation Factor 2, Endoplasmin, ER ATPase, Ezrin, Fibronectin, Folate receptor alpha, Gelsolin, Hyaluronan and proteoglycan link protein, Immunoglobulin J chain, Keratin 2, Keratin 5, Keratin 6A, Keratin 14, Keratin 16, Keratin 17, Lactoperoxidase, Lanosterol Synthase, Lysozyme C, Mucin-1, Mucin-4, Neutral alpha-glucosidase, Peptidyl-prolyl cis-trans isomerase A, Peroxiredoxin-6, Protein disulfide isomerase, Serum Albumin, Sulfhydryl oxidase 1, Syntaxin binding protein 2, Tenascin, Toll-like receptor 2, and Vitronectin.

In one embodiment, the microvesicles are exosomes or globules.

In one embodiment, the sample is selected from the group consisting of breastmilk, urine, saliva and plasma.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein the terms “alteration,” “defect,” “variation,” or “mutation,” refers to a mutation in a gene in a cell that affects the function, activity, expression (transcription or translation) or conformation of the polypeptide that it encodes. Mutations encompassed by the present invention can be any mutation of a gene in a cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing the mRNA (splice site mutation) or cause a shift in the reading frame (frameshift).

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic animals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “amplification” refers to the operation by which the number of copies of a target nucleotide sequence present in a sample is multiplied.

By the term “applicator,” as the term is used herein, is meant any device including, but not limited to, a hypodermic syringe, a pipette, an iontophoresis device, a patch, and the like, for administering the compositions of the invention to a subject.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “marker” or “biomarker” is meant to include a parameter which is useful according to this invention for determining the presence and/or severity of diabetes.

The level of a marker or biomarker “significantly” differs from the level of the marker or biomarker in a reference sample if the level of the marker in a sample from the patient differs from the level in a sample from the reference subject by an amount greater than the standard error of the assay employed to assess the marker, and preferably at least 10%, and more preferably 25%, 50%, 75%, or 100%.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between than a control.

“Differentially decreased expression” or “down regulation” refers to biomarker product levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments there between than a control.

A “disease” is a state of health of an animal wherein the animal cannot maintain homeostasis, and wherein if the disease is not ameliorated then the animal's health continues to deteriorate.

“Lactation insufficiency,” as used herein is a condition wherein a subject produces breast milk of sub-optimal composition or produces a low volume of breast milk, either of which may not match the needs of their infant. Lactation insufficiency can include lactation failure, but does not have to be so detrimental as to cause overt lactation failure.

As used herein, an “immunoassay” refers to a biochemical test that measures the presence or concentration of a substance in a sample, such as a biological sample, using the reaction of an antibody to its cognate antigen, for example the specific binding of an antibody to a protein. Both the presence of the antigen or the amount of the antigen present can be measured.

As used herein, an “instructional material” includes a publication, a recording, a diagram, or any other medium of expression which can be used to communicate the usefulness of a component of the invention in a kit for detecting biomarkers disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The “level” of one or more biomarkers means the absolute or relative amount or concentration of the biomarker in the sample.

The term “marker (or biomarker) expression” as used herein, encompasses the transcription, translation, post-translation modification, and phenotypic manifestation of a gene, including all aspects of the transformation of information encoded in a gene into RNA or protein. By way of non-limiting example, marker expression includes transcription into messenger RNA (mRNA) and translation into protein, as well as transcription into types of RNA such as transfer RNA (tRNA), ribosomal RNA (rRNA), and microRNA that are not translated into protein.

The terms “microarray” and “array” refers broadly to both “DNA microarrays” and “DNA chip(s),” and encompasses all art-recognized solid supports, and all art-recognized methods for affixing nucleic acid molecules thereto or for synthesis of nucleic acids thereon. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes. Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.) Arrays may be packaged in such a manner as to allow for diagnostic use or can be an all-inclusive device; e.g., U.S. Pat. Nos. 5,856,174 and 5,922,591 incorporated in their entirety by reference for all purposes. Arrays are commercially available from, for example, Affymetrix (Santa Clara, Calif.) and Applied Biosystems (Foster City, Calif.), and are directed to a variety of purposes, including genotyping, diagnostics, mutation analysis, marker expression, and gene expression monitoring for a variety of eukaryotic and prokaryotic organisms. The number of probes on a solid support may be varied by changing the size of the individual features. In one embodiment the feature size is 20 by 25 microns square, in other embodiments features may be, for example, 8 by 8, 5 by 5 or 3 by 3 microns square, resulting in about 2,600,000, 6,600,000 or 18,000,000 individual probe features.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

By the term “modulating,” as used herein, is meant mediating a detectable increase or decrease in the activity and/or level of a mRNA, polypeptide, or a response in a subject compared with the activity and/or level of a mRNA, polypeptide or a response in the subject in the absence of a treatment or compound, and/or compared with the activity and/or level of a mRNA, polypeptide, or a response in an otherwise identical but untreated subject.

A “lactation sufficient” or “normal” subject does not have any form of lactation insufficiency.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “providing a prognosis” refers to providing a prediction of the probable course and outcome of lactation insufficiency, including prediction of severity, affect on infant, etc. The methods can also be used to devise a suitable therapeutic plan.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

“Sample” or “biological sample” as used herein means a biological material isolated from an individual, including but is not limited to organ, tissue, exosome, breast milk, blood, plasma, saliva, urine and other body fluid. The biological sample may contain any biological material suitable for detecting the desired biomarkers, and may comprise cellular and/or non-cellular material obtained from the individual.

“Standard control value” as used herein refers to a predetermined amount of a particular protein or nucleic acid that is detectable in a biological sample. The standard control value is suitable for the use of a method of the present invention, in order for comparing the amount of a protein or nucleic acid of interest that is present in a biological sample. An established sample serving as a standard control provides an average amount of the protein or nucleic acid of interest in the biological that is typical for an average, healthy person of reasonably matched background, e.g., gender, age, ethnicity, and medical history. A standard control value may vary depending on the protein or nucleic acid of interest and the nature of the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based on the identification of biological sample-based biomarkers that can identify lactation insufficiency in a subject.

In one embodiment, the invention provides a biomarker for the detection of lactation insufficiency. In one embodiment, the biomarker for the detection of lactation insufficiency includes but is not limited to vitronectin, folate receptor alpha, peroxiredoxin-6, mucin-4, elongation factor 2, cytosolic 10-F-D, and endoplasmin.

In other embodiments, the biomarker is selected from 14-3-3 protein, 40S ribosomal protein, 60S ribosomal protein, 60S ribosomal protein L3, 60S ribosomal protein L5, 60S ribosomal protein L7, Annexin A2, Apolipoprotein E, Carbonic Anhydrase, CD14, Cell division control protein 42, Chordin-like protein 2, Complement C3, Cytosolic 10-F-D, Elongation Factor 1, Elongation Factor 2, Endoplasmin, ER ATPase, Ezrin, Fibronectin, Folate receptor alpha, Gelsolin, Hyaluronan and proteoglycan link protein, Immunoglobulin J chain, Keratin 2, Keratin 5, Keratin 6A, Keratin 14, Keratin 16, Keratin 17, Lactoperoxidase, Lanosterol Synthase, Lysozyme C, Mucin-1, Mucin-4, Neutral alpha-glucosidase, Peptidyl-prolyl cis-trans isomerase A, Peroxiredoxin-6, Protein disulfide isomerase, Serum Albumin, Sulfhydryl oxidase 1, Syntaxin binding protein 2, Tenascin, Toll-like receptor 2, and Vitronectin.

### Identifying a Marker or Biomarker

The invention includes methods for the identification of differentially expressed markers between samples of lactation sufficient and lactation insufficient subjects.

The invention contemplates the identification of differentially expressed markers by whole genome nucleic acid microarray, to identify markers differentially expressed between lactation sufficient and lactation insufficient subjects. The invention further contemplates using methods known to those skilled in the art to detect and to measure the level of differentially expressed marker expression products, such as RNA, microRNA and protein, to measure the level of one or more differentially expressed marker expression products.

Methods of detecting or measuring gene expression may utilize methods that focus on cellular components (cellular examination), or methods that focus on examining extracellular components (fluid examination). Because gene expression involves the ordered production of a number of different molecules, a cellular or fluid examination may be used to detect or measure a variety of molecules including RNA, microRNA, protein, and a number of molecules that may be modified as a result of the protein's function. Typical diagnostic methods focusing on nucleic acids include amplification techniques such as PCR and RT-PCR (including quantitative variants), and hybridization techniques such as in situ hybridization, microarrays, blots, and others. Typical diagnostic methods focusing on proteins include binding techniques such as ELISA, immunohistochemistry, microarray and functional techniques such as enzymatic assays.

The genes identified as being differentially expressed may be assessed in a variety of nucleic acid detection assays to detect or quantify the expression level of a gene or multiple genes in a given sample. For example, traditional Northern blotting, nuclease protection, RT-PCR, microarray, and differential display methods may be used for detecting gene expression levels. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays are most efficiently designed with array or chip hybridization-based methods for detecting the expression of a large number of genes. Any hybridization assay format may be used, including solution-based and solid support-based assay formats.

The protein products of the genes identified herein can also be assayed to determine the amount of expression. Methods for assaying for a protein include Western blot, immunoprecipitation, and radioimmunoassay. The proteins analyzed may be localized intracellularly (most commonly an application of immunohistochemistry) or extracellularly (most commonly an application of immunoassays such as ELISA).

Biological samples may be of any biological tissue or fluid. Frequently the sample will be a “clinical sample” which is a sample derived from a patient.

Controls groups may either be normal or samples from known lactation insufficiency. As described below, comparison of the expression patterns of the sample to be tested with those of the controls can be used to diagnose between normal and lactation insufficient subjects. In some instances, the control groups are only for the purposes of establishing initial cutoffs for the assays of the invention. Therefore, in some instances, the systems and methods of the invention can diagnose between normal and lactation insufficient subjects without the need to compare with a control group.

### Methods of Diagnosis

The present invention relates to the identification of biomarkers associated with lactation insufficiency. Accordingly, the present invention features methods for identifying subjects who are at risk of developing lactation insufficiency, including those subjects who are asymptomatic or only exhibit non-specific indicators of lactation insufficiency by detection of the biomarkers disclosed herein.

The invention provides improved diagnosis of lactation insufficiency. The risk of having lactation insufficiency can be assessed by measuring one or more of the biomarkers described herein, and comparing the measured values to reference or index values. Such a comparison can be undertaken with mathematical algorithms or formula in order to combine information from results of multiple individual biomarkers and other parameters into a single measurement or index.

The biomarkers of the present invention can thus be used to generate a biomarker profile or signature of subjects: (i) who do not have and are not expected to develop lactation insufficiency and/or (ii) who have or expected to develop lactation insufficiency. The biomarker profile of a subject can be compared to a predetermined or reference biomarker profile to diagnose or identify subjects at risk for lactation insufficiency. Data concerning the biomarkers of the present invention can also be combined or correlated with other data or test results, such as, without limitation, measurements of clinical parameters or other algorithms for lactation insufficiency. Other data includes age, ethnicity, body mass index (BMI), total cholesterol levels, genetic variants, and milk sodium levels. The machine-readable media can also comprise subject information such as medical history and any relevant family history.

In various embodiments, methods are disclosed herein that may be of use to determine whether a subject has lactation insufficiency. In some embodiments, these methods may utilize a biological sample (such as breastmilk, urine, saliva or plasma), for the detection of one or more markers of the invention in the sample.

In one embodiment, the invention provides a biomarker for the detection of lactation insufficiency. In one embodiment, the biomarker for the detection of lactation insufficiency includes but is not limited to vitronectin, folate receptor alpha, peroxiredoxin-6, mucin-4, elongation factor 2, cytosolic 10-F-D, and endoplasmin.

In some embodiments, the biomarker is selected from 14-3-3 protein, 40S ribosomal protein, 60S ribosomal protein, 60S ribosomal protein L3, 60S ribosomal protein L5, 60S ribosomal protein L7, Annexin A2, Apolipoprotein E, Carbonic Anhydrase, CD14, Cell division control protein 42, Chordin-like protein 2, Complement C3, Cytosolic 10-F-D, Elongation Factor 1, Elongation Factor 2, Endoplasmin, ER ATPase, Ezrin, Fibronectin, Folate receptor alpha, Gelsolin, Hyaluronan and proteoglycan link protein, Immunoglobulin J chain, Keratin 2, Keratin 5, Keratin 6A, Keratin 14, Keratin 16, Keratin 17, Lactoperoxidase, Lanosterol Synthase, Lysozyme C, Mucin-1, Mucin-4, Neutral alpha-glucosidase, Peptidyl-prolyl cis-trans isomerase A, Peroxiredoxin-6, Protein disulfide isomerase, Serum Albumin, Sulfhydryl oxidase 1, Syntaxin binding protein 2, Tenascin, Toll-like receptor 2, and Vitronectin. In one embodiment, the method comprises detecting one or more markers in a biological sample of the subject. Preferably, the biological sample is breastmilk. In various embodiments, the level of one or more of markers of the invention in the biological sample of the subject is compared with the level of a corresponding biomarker in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

In various embodiments, the subject is a human subject, and may be of any race or age.

Information obtained from the methods of the invention described herein can be used alone, or in combination with other information (e.g., disease status, disease history, vital signs, blood chemistry, etc.) from the subject or from the biological sample obtained from the subject.

In other various embodiments of the methods of the invention, the level of one or more markers of the invention is determined to be increased when the level of one or more of the markers of the invention is increased by at least 10%, by at least 20%, by at least 30%, by at least 40%, by at least 50%, by at least 60%, by at least 70%, by at least 80%, by at least 90%, or by at least 100%, when compared to with a comparator control.

In the methods of the invention, a biological sample from a subject is assessed for the level of one or more of the markers of the invention in the biological sample obtained from the patient. The level of one or more of the markers of the invention in the biological sample can be determined by assessing the amount of polypeptide of one or more of the biomarkers of the invention in the biological sample, the amount of mRNA of one or more of the biomarkers of the invention in the biological sample, the amount of microRNA that regulates of one or more of the biomarkers of the invention in the biological sample, the amount of enzymatic activity of one or more of the biomarkers of the invention in the biological sample, or a combination thereof.

### Detecting a Biomarker

In one embodiment, the invention includes detecting a biomarker mRNA or microRNA in a microvesicles, wherein the bodily fluid is breastmilk and the microvesicles are exosomes. Detecting exosomal biomarkers is in particular performed in a portion of breastmilk.

In one embodiment, detecting biomarkers is performed in a bodily fluid, breastmilk, urine, or saliva, that meets the demands of an inexpensive, non-invasive and accessible bodily fluid to act as an ideal medium for investigative analysis.

Biomarkers generally can be measured and detected through a variety of assays, methods and detection systems known to one of skill in the art. Various methods include but are not limited to refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). See also PCT Publications WO/2004/056456 and WO/2004/088309. In this regard, biomarkers can be measured using the above-mentioned detection methods, or other methods known to the skilled artisan. Other biomarkers can be similarly detected using reagents that are specifically designed or tailored to detect them.

Different types of biomarkers and their measurements can be combined in the compositions and methods of the present invention. In various embodiments, the protein form of the biomarkers is measured. In various embodiments, the nucleic acid form of the biomarkers is measured. In exemplary embodiments, the nucleic acid form is mRNA or microRNA. In various embodiments, measurements of protein biomarkers are used in conjunction with measurements of nucleic acid biomarkers.

Methods for detecting mRNA, such as RT-PCR, real time PCR, branch DNA, NASBA and others, are well known in the art. Using sequence information provided by the database entries for the biomarker sequences, expression of the biomarker sequences can be detected (if present) and measured using techniques well known to one of ordinary skill in the art. For example, sequences in sequence database entries or sequences disclosed herein can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and reference populations can be made by comparing relative amounts of the examined DNA sequences in the test and reference cell populations. In addition to Northern blot and RT-PCR, RNA can also be measured using, for example, other target amplification methods (e.g., TMA, SDA, NASBA), signal amplification methods (e.g., bDNA), nuclease protection assays, in situ hybridization and the like.

The concentration of the biomarker in a sample may be determined by any suitable assay. A suitable assay may include one or more of the following methods, an enzyme assay, an immunoassay, mass spectrometry, chromatography, electrophoresis or an antibody microarray, or any combination thereof. Thus, as would be understood by one skilled in the art, the system and methods of the invention may include any method known in the art to detect a biomarker in a sample.

The invention described herein also relates to methods for a multiplex analysis platform. In one embodiment, the method comprises an analytical method for multiplexing analytical measurements of markers. In another embodiment, the method comprises a set of compatible analytical strategies for multiplex measurements of markers and/or metabolites in biological samples.

### Kits

The present invention also pertains to kits useful in the methods of the invention. Such kits comprise various combinations of components useful in any of the methods described elsewhere herein, including for example, materials for quantitatively analyzing a biomarker of the invention (e.g., polypeptide and/or nucleic acid), materials for assessing the activity of a biomarker of the invention (e.g., polypeptide and/or nucleic acid), and instructional material. For example, in one embodiment, the kit comprises components useful for the quantification of a desired nucleic acid in a biological sample. In another embodiment, the kit comprises components useful for the quantification of a desired polypeptide in a biological sample. In a further embodiment, the kit comprises components useful for the assessment of the activity (e.g., enzymatic activity, substrate binding activity, etc.) of a desired polypeptide in a biological sample.

In a further embodiment, the kit comprises the components of an assay for monitoring the effectiveness of a treatment administered to a subject in need thereof, containing instructional material and the components for determining whether the level of a biomarker of the invention in a biological sample obtained from the subject is modulated during or after administration of the treatment. In various embodiments, to determine whether the level of a biomarker of the invention is modulated in a biological sample obtained from the subject, the level of the biomarker is compared with the level of at least one comparator control contained in the kit, such as a positive control, a negative control, a historical control, a historical norm, or the level of another reference molecule in the biological sample. In certain embodiments, the ratio of the biomarker and a reference molecule is determined to aid in the monitoring of the treatment.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Methods and Diagnostics for Diagnosing Lactation Sufficiency

The results presented herein demonstrate the discovery of breastmilk exosomal biomarkers capable of diagnosing lactation deficiency in subjects. The markers of the invention can be used to screen, assess risk, and diagnose lactation deficiency prior to the point in which infant health is compromised or they are prematurely weaned.

Breast milk was obtained from women. From the breast milk, exosomes were isolated and purified and the protein signature of these vesicles was analyzed using mass spectrometry. The protein signature in 4 groups of women was compared; women with high milk sodium (a hallmark of breast dysfunction; Group A), women with two different genetic variants in a protein that we recently showed impaired breast function (Group B and C), and women with low milk sodium and no genetic variation (controls; Group D). The “exosome protein signatures” (the 40 most abundant proteins) from these women were unique. For example women in Group D who presumably had “healthy” breast tissue, uniquely expressed vitronectin, folate receptor alpha and peroxiredoxin-6; vitronectin is a cell adhesion molecule, folate receptor alpha takes up folate from circulation and peroxiredoxin-6 is a powerful reductant enzyme. In contrast, women in Groups A, B, and C, who had breast “dysfunction” of different etiologies all had enhanced expression of mucin-4, elongation factor 2, cytosolic 10-F-D, and endoplasmin. Mucin-4 is an anti-adhesive molecule, cytosolic 10-F-D catalyzes 10-formyltetrahydrofolate→tetrahydrofolate creating purines for DNA synthesis, elongation factor 2 catalyzes protein synthesis, and endoplasmin is an ER chaperone activated by ER stress. Based on the current understanding of mammary gland biology, it is reasonable that these former proteins might be enriched in the healthy breast, while the latter proteins may be enhanced in the dysfunctional breast.

Four non-limiting molecules found in milk exosomes were chosen for a pilot study to provide proof of concept (vitronectin and peroxiredxin-6 as “healthy” predictors and mucin-4 and endoplasmin as “dysfunctional” predictors). The concentration of these 4 molecules was measured in a subset of milk samples with high sodium that were previously banked and it was determined that there is an association between their abundance and other indices of breast function. Next, a clinical study determines if any of these molecules are associated with the onset of lactation, which has been used as an index of lactation success, milk volume or milk sodium levels in breastfeeding women to determine if these molecules can predict breast dysfunction. These milk samples are banked and used for analysis of further molecular targets as needed. In addition, urine and saliva is collected, and exosomes are isolated and used for proteomic profiling to identify differences in exosomal proteins between women with sufficient and sub-optimal lactation.

Two different multiplex platforms are used to diagnose lactation (in)sufficiency. First, microfluidics-based “lab-on-a-chip” is used as a point-of-care device, which isolates exosomes, detects and quantifies a selection of specific proteins that in combination will diagnose lactation insufficiency. Second, is a lateral flow “dipstick” device, which can be used by individual consumers in the home. The lateral flow device detects and semi-quantifies selection of specific proteins that alerts a woman to the fact that she may have lactation insufficiency and should see her healthcare provider.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of diagnosing a lactation performance of a subject, the method comprising:
a. isolating microvesicles in a biological sample of the subject,
b. determining the level of a biomarker in the microvesicles,
c. comparing the level of the biomarker in the biological sample of the subject with a comparator control, and
d. diagnosing the lactation performance of the subject to be lactation insufficient if the level of the biomarker in the biological sample of the subject is altered at a statistically significant amount when compared with the level of the biomarker of the comparator control.

2. The method of claim 1, wherein the microvesicles are selected from the group consisting of exosomes and globules.

3. The method of claim 1, wherein the biological sample is selected from the group consisting of breastmilk, urine, saliva and plasma.

4. The method of claim 1, wherein the biomarker is selected from the group consisting of vitronectin, folate receptor alpha, peroxiredoxin-6, mucin-4, elongation factor 2, cytosolic 10-F-D, endoplasmin, and any combination thereof.

5. The method of claim 1, wherein the lactation insufficiency is selected from the group consisting of low volume, sub-optimal composition, and a combination thereof.

6. The method of claim 1, wherein the comparator control is the level of the biomarker in the biological sample of a lactation sufficient subject.

7. The method of claim 1, wherein the level of the biomarker in the biological sample of subject is increased at a statistically significant amount when compared with the level of the biomarker of the comparator control.

8. The method of claim 1, wherein the level of the biomarker in the biological sample of subject is decreased at a statistically significant amount when compared with the level of the biomarker of the comparator control.

9. The method of claim 1, wherein the level of the biomarker in the biological sample is determined by measuring the level of mRNA of the biomarker in the biological sample.

10. The method of claim 1, wherein the level of the biomarker in the biological sample is determined by measuring the level of polypeptide of the biomarker in the biological sample.

11. The method of claim 1, wherein the comparator control is at least one selected from the group consisting of: a positive control, a negative control, a historical control, a historical norm, or the level of a reference molecule in the biological sample.

12. (canceled)

13. (canceled)

14. A system for diagnosing lactation performance, wherein the system detects the level of a biomarker in microvesicles isolated from a biological sample obtained from a subject, wherein the of the biomarker diagnoses lactation insufficiency in the subject.

15. The system of claim 14, wherein the microvesicles are selected from the group consisting of exosomes and globules.

16. The system of claim 14, wherein the biological sample is selected from the group consisting of breastmilk, urine, saliva and plasma.

17. The system of claim 14, wherein the biomarker is selected from the group consisting of vitronectin, folate receptor alpha, peroxiredoxin-6, mucin-4, elongation factor 2, cytosolic 10-F-D, endoplasmin, and any combination thereof.

18. (canceled)

19. The system of claim 14, wherein the reagent is selected from the group consisting of a microfluidics chip and a lateral flow test.

20. (canceled)

21. A kit for diagnosing lactation performance, the kit comprising a reagent for measuring the level of a biomarker in microvesicles isolated from a biological sample of a subject.

22. The kit of claim 21, wherein the biomarker is selected from the group consisting of vitronectin, folate receptor alpha, peroxiredoxin-6, mucin-4, elongation factor 2, cytosolic 10-F-D, endoplasmin, and any combination thereof.

23. The kit of claim 21, wherein the microvesicles are selected from the group consisting of exosomes and globules.

24. The kit of claim 21, wherein the biological sample is selected from the group consisting of breastmilk, urine, saliva and plasma.

25. (canceled)

26. (canceled)
